(19) United States
(12) Patent Application Publication
(10) Pub. No.: US 2003/0185782 A1
(43) Pub. Date: Oct. 2, 2003

(54) Cosmetic composition comprising a pro-adhesive material and a diffusing compound, which are separated

(76) Inventors: Frederic Auguste, Chevilly-Larue (FR); Beatrice Toumi, Verrieres Ie Buisson (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/347,475

(22) Filed: Jan. 21, 2003

Related U.S. Application Data
(60) Provisional application No. 60/354,923, filed on Feb. 11, 2002.

(30) Foreign Application Priority Data
Jan. 18, 2002 (FR) 02 00630

(51) Int. Cl. A61K 7/06; A61K 7/11

(52) U.S. Cl. 424/70.11; 424/70.12; 424/70.17; 424/70.16

(57) ABSTRACT

A cosmetic and/or physiological composition comprising at least one pro-adhesive material and at least one diffusing compound, wherein a system created by interaction of the pro-adhesive material and the diffusing compound is an adhesive system, and wherein the composition further comprises at least one medium for separating the pro-adhesive material and the diffusing compound, wherein the at least one medium limits the molecular diffusion between the pro-adhesive material and the diffusing compound within the composition, and wherein the weight ratio between the pro-adhesive material and the diffusing compound ranges from 0.01 to 10, such as from 0.05 to 2 and further such as from 0.1 to 2, further wherein the composition can possess enhanced transfer-resistance and/or long-lasting properties.

This application claims benefit of U.S. Provisional Application No. 60/354,923, filed Feb. 11, 2002.

Disclosed herein is a composition comprising at least one pro-adhesive material, at least one diffusing compound, and at least one medium that separates the pro-adhesive material and the diffusing compound within said composition, intended for example, to be used in the fields of cosmetics and hygiene. Further disclosed herein is, for example, a along-lasting and transfer-resistant makeup composition and/or care composition for both human facial skin and body skin, and/or for integuments.

Cosmetic compositions, for example, makeup compositions such as lipsticks, foundations, body makeup products, concealer products, eyeshadows or powders, generally comprise fatty substances such as oils and/or waxes, and a particulate phase generally composed of fillers and pigments. They may thus be in the form of an anhydrous gel, in the form of a stick or tube or in the form of a soft paste, for example, certain foundations, eyeshadows or lipsticks. They may also be in the form of a powder, which may be, for example, free, compacted or pressed. Makeup compositions may also comprise water or a hydrophilic phase, and may then, for example, be in the form of an oil-in-water emulsion, a water-in-oil emulsion or a multiple emulsion, such as when the composition is a foundation, a tinted cream, a care cream or an antisun product. Nail varnishes are generally in the form of a solution of an organic solvent.

These compositions do not always show good staying power when they are applied to the skin, mucous membranes or semi-mucous membranes. For example, it has been found that certain compositions have a tendency to travel within the wrinkles and/or fine lines of the skin, in the case of foundations; in the fine lines around the lips, in the case of lipsticks; in the folds of the eyelids, in the case of eyeshadows. The appearance of lines in the makeup generated by the movements of the eyelids has also been found, especially in the case of eyeshadows. Similarly, the original colour is liable to change over time. In the case of nail varnishes, the product is liable to crack, flake or fail to withstand friction.

Moreover, certain makeup products may have the drawback of transferring. This means that the composition is liable to become at least partially deposited onto certain supports with which it comes into contact, for example, a glass, an item of clothing or the skin. On becoming deposited, the composition may leave a mark on the support. This transferring therefore results in mediocre persistence of the composition on the skin or mucous membranes, resulting in the need to renew its application regularly. Moreover, the appearance of unacceptable marks on certain items of clothing, and especially on blouse collars, may preclude some women from using this type of makeup.

These phenomena can all result in an unsightly effect that it would obviously be desirable to avoid.

There is thus still a need for products, such as makeup products, which have enhanced staying power while having good cosmetic properties, such as properties of sliding on application, and also such as softness, moisturization and comfort qualities after applying the makeup.

The inventors have found, entirely surprisingly, that the presence of at least one pro-adhesive material, at least one diffusing compound, and at least one medium for separating the pro-adhesive material and the diffusing compound, in a cosmetic and/or physiological composition can produce a makeup with enhanced staying power, that transfers little, if at all.

Disclosed herein is thus a cosmetic and/or physiological composition comprising:

i) at least one pro-adhesive material that satisfies the following condition, for any frequency ranging from 2×10⁻² to 2 Hz:

10⁸ Pa≧G′(35° C.)≧2×10⁴ Pa,

such as:

10⁸ Pa≧G′(35° C.)≧10⁵ Pa,

further such as:

5×10⁷ Pa≧G′(35° C.)≧3×10⁵ Pa,

and even further such as:

10⁷ Pa≧G′(35° C.)≧3×10⁵ Pa,

wherein:

G′(35° C.) is the elastic shear modulus of the at least one pro-adhesive material, measured at a temperature of 35° C., and

ii) at least one diffusing compound,

wherein a system created by the interaction of the at least one pro-adhesive material and the at least one diffusing compound is an adhesive system that satisfies the following conditions:

G′_{AS}(2 Hz, 35° C.)≧10³ Pa, and

G′_{AS}(2×10⁻² Hz, 35° C.)≦3×10⁵ Pa,

wherein:

G′_{AS}(2 Hz, 35° C.) is the elastic shear modulus of the adhesive system, measured at a frequency of 2 Hz and at a temperature of 35° C., and

G′_{AS}(2×10⁻² Hz, 35° C.) is the elastic shear modulus of the adhesive system, measured at a frequency of 2×10⁻² Hz and at a temperature of 35° C.; and

iii) at least one medium for separating the at least one pro-adhesive material and the at least one diffusing compound, wherein the at least one medium limits the molecular diffusion between the at least one pro-adhesive material and the at least one diffusing compound within the composition, and wherein the weight ratio between the at least one pro-adhesive material and the at least one diffusing compound ranges from 0.01:1 to 10:1, such as from 0.05:1 to 2:1 and further such as from 0.1:1 to 2:1.

Also disclosed herein is a a cosmetic and/or physiological composition, to be used on a keratin material, comprising:

i) at least one pro-adhesive material that satisfies the following condition, for any frequency ranging from 2×10⁻² to 2 Hz:

10⁸ Pa≧G′(35° C.)≧2×10⁴ Pa,

such as:

10⁸ Pa≧G′(35° C.)≧10⁵ Pa,

further such as:

5×10⁷ Pa≧G′(35° C.)≧3×10⁵ Pa,

and even further such as:

10⁷ Pa≧G′(35° C.)≧3×10⁵ Pa,

wherein:

G′(35° C.) is the elastic shear modulus of the said pro-adhesive material, measured at a temperature of 35° C., and

ii) at least one diffusing compound, wherein a system created by the interaction of the at least one pro-adhesive material and the at least one diffusing compound is an adhesive system that satisfies the following conditions:

G′_{AS}(2 Hz, 35° C.)≧10³ Pa, and

G′_{AS}(2×10⁻² Hz, 35° C.)≦3×10⁵ Pa,

wherein:

G′_{AS}(2 Hz, 35° C.) is the elastic shear modulus of the adhesive system, measured at a frequency of 2 Hz and at a temperature of 35° C., and

G′_{AS}(2×10⁻² Hz, 35° C.) is the elastic shear modulus of the adhesive system, measured at a frequency of 2×10⁻² Hz and at a temperature of 35° C.; and

iii) at least one medium for separating the at least one pro-adhesive material and the at least one diffusing compound, wherein the at least one medium limits the molecular diffusion between the at least one pro-adhesive material and the at least one diffusing compound within the composition, and wherein the weight ratio between the at least one pro-adhesive material and the at least one diffusing compound ranges from 0.01:1 to 10:1, such as from 0.05:1 to 2:1 and further such as from 0.1:1 to 2:1, such that, during or after application, said interaction can take place.

The term “interaction” means that the at least one diffusing compound has molecularly diffused into the at least one pro-adhesive material. Molecular diffusion is, by definition (Atkins, Physical Chemistry, 5th edition, 1995, p. 846), the migration of a material due to the effect of a concentration gradient.

The expression “limitation of the molecular diffusion” means that the at least one diffusing compound has not been able, in the composition and before application, to diffuse into the at least one pro-adhesive, generally in an amount such that the weight ratio between the pro-adhesive material and the diffusing compound is less than 10:1, and/or such as the elastic shear modulus of the pro-adhesive material comprising the diffusing compound is modified by more than about 30% relative to the shear modulus of the pure pro-adhesive material. Another way of measuring said limitation may be to evaluate the level of aggregation of a pulverulent composition: it should be virtually constant over time after manufacture of the composition. It is also possible to use another measurement that is suitable according to the nature of the composition, as may be judged by a person skilled in the art.

The term “application” means the introduction of the product onto a keratin material by a user.

The composition disclosed herein can have enhanced staying power properties on the skin and excellent “transfer-resistance” properties. It can adhere well to the skin. It can be resistant to friction. It can have very good makeup behaviour, i.e., it can allow a homogeneous deposition over time and a uniform makeup and/or care can result. Once applied to the skin, it does not migrate and can have enhanced colour fastness over time. The composition can spread easily and can be very comfortable to wear.

The medium capable of limiting the molecular diffusion between the at least one pro-adhesive material and the at least one diffusing compound within the composition can allow the pro-adhesive material/the diffusing compound interaction essentially during application or after application. The system comprising the pro-adhesive material and the diffusing compound that have interacted can show adhesive properties once they are placed in contact after or during application, and once the diffusing compound has molecularly diffused into the pro-adhesive material.

Advantageously, compared with a case in which the diffusing compound and the pro-adhesive material are present in two different cosmetic and/or physiological compositions, such as those present in the same care and/or makeup kit, which the user must apply successively in order to bring about the interaction between the pro-adhesive material and the diffusing compound directly on any keratin material or substrate, the presence of said at least one medium can allow the composition as disclosed herein to be applied to any keratin material, e.g., to the skin and/or the integuments, in a single application, which can avoid the need to perform two care and/or makeup actions.

Further disclosed herein is the use, in or for the manufacture of a cosmetic and/or physiological composition, comprising at least one pro-adhesive material, at least one diffusing compound and at least one medium, to achieve at least one desirable effect chosen from reducing and preventing transfer and migration of the composition deposited on a keratin material towards a support placed in contact with the deposit, and enhancing the staying power over time of the resulting makeup and/or care product (i.e., increasing the strength of adhesion of the care and/or makeup composition to the keratin material over time).

Also disclosed herein is the use, in or for the manufacture of a cosmetic and/or physiological composition, comprising at least one pro-adhesive material, at least one diffusing compound and at least one medium as defined above to increase the colour fastness of the composition over time.

Further disclosed herein is a non-therapeutic care and/or makeup process for at least one surface chosen from human skin, mucous membranes, semi-mucous membranes and integuments, comprising applying to said at least one surface a pulverulent cosmetic composition as defined above.

The term “keratin material” means any cutaneous area of the body and/or face, such as the skin, including mucous membranes and semi-mucous membranes, including the lips, and the integuments. The integuments include, for example, the eyelashes, the eyebrows, the nails and the hair, and also any substitute for these materials, for instance false nails, wigs, false eyelashes and hairpieces.

The composition disclosed herein can, for example, find an advantageous application in the field of care and/or makeup of the skin, including mucous membranes and semi-mucous membranes, and the integuments. The term “mucous membrane” means, for example, the inner part of the lower eyelid; among semi-mucous membranes are the lips. Thus, the composition disclosed herein can find, for example, an application in the field of care and/or makeup products such as for the lips, the face and the skin, such as foundations, concealer products, eyeshadows, powders, body makeup products, lipsticks, self-tanning products and antisun products, and in the field of care and/or makeup products for the integuments (and for substitutes thereof, for instance false nails and false eyelashes) such as mascaras and nail varnishes.

As disclosed herein, the expression “physiological composition” means a non-toxic physiologically acceptable composition that may be applied to human skin and integuments. The expression “cosmetic composition” means a composition of pleasant appearance, odour and feel.

The disclosed composition comprises at least one medium capable of limiting the molecular diffusions between the at least one pro-adhesive material and the at least one diffusing compound within the composition, and capable of allowing the diffusions during application or after application to a substrate. Thus, the at least one medium can serve to virtually prevent molecular diffusions at least between the diffusing compound and the pro-adhesive material within the composition.

The medium that can limit molecular diffusions between the at least one pro-adhesive material and the at least one diffusing compound in the composition and that can allow the diffusions during application or after application may be chosen, for example, from monolayers and multilayers of amphiphilic molecules (thus forming, for example, simple or multiple emulsions, or liposomes) that are solid at room temperature (about 25° C.) and layers of polymers, (thus forming, for example, nanocapsules). The choice of said medium depends both on the nature of the at least one pro-adhesive material and the at least one diffusing compound (e.g., hydrophobicity or hydrophilicity) and on the presentation form of the composition.

On application or after application, the at least one pro-adhesive material and the at least one diffusing compound are placed in contact and can interact by modification of the medium that limited the diffusions in the composition. Such a modification may be, for example, and in a non-limiting manner, of three kinds, depending on the chemical nature and the organization of the medium, which is capable of limiting the molecular diffusions between the at least one pro-adhesive material and the at least one diffusing compound in the composition and is capable of allowing the diffusions during or after application:

by the effect of stresses chosen from thermal and mechanical stresses associated with the action of applying the composition and/or the temperature of the keratin material or the substrate. It is then possible for there to be rupture of the medium separating the constituents (for example, breaking of an emulsion), melting of the aliphatic portions constituting this medium (e.g., melting of the surfactants, resulting in permeability to the diffusing compound in certain emulsions), and cracking associated with a temperature-related phase separation within liposomes containing poly(N-isopropylacrylamides) (Francoise Winnik, “Polymer-stabilized liposomes: design, characterization and applications”, presentation at Formulations Forum '99, Orlando, March 1999);

by the effect of a modification of the medium associated with the evaporation of at least one volatile solvent, such as the evaporation of water from, for example, triple emulsions and liposomes; and

by the effect of the chemical degradation of the medium, for example, in the case of a polymer that constitutes nanocapsules, such as that disclosed in EP 0 447 318 A1.

The composition disclosed herein comprises at least one pro-adhesive material. As disclosed herein, the term “material” means a polymer or a polymer system, such as an at least partially non-crosslinked polymer or polymer system, further such as a virtually non-crosslinked polymer or polymer system, which may comprise one or more polymers of different nature. Further as disclosed herein, the term “pro-adhesive” means capable of becoming adhesive generally by interaction with a diffusing compound. The term “adhesive” means a substance that adheres to a surface, i.e., after contact between a finger, such as the index finger, and the surface of the composition containing the material, there is a sensation of stickiness of the surface when the contact therewith is broken by removing the finger, in the same way as may be experienced after contact with an adhesive surface, for example, an adhesive tape. On contact or after contact with the skin, the composition disclosed herein can generally stick to the surface of the skin and can thus have enhanced staying power; thus, the resulting composition on the skin will generally not have any surface stickiness, i.e., after contact between the finger and the surface of the product, there is no sensation of stickiness of the surface when the contact therewith is broken by removing the finger.

The viscoelastic properties of a material are conventionally defined by two characteristic values, which are as follows:

the elastic shear modulus, which represents the elastic behaviour of the material for a given frequency and which is conventionally written as G′,

the viscous shear modulus, which represents the viscous behaviour of the material for a given frequency, and which is conventionally written as G″.

These characteristic values are, for example, defined in the “Handbook of Pressure Sensitive Adhesive Technology” 3rd Edition, D. Satas, Chapter 9, pp. 155 to 157.

These viscoelastic properties are measured during dynamic tests under sinusoidal stresses of low amplitude (small deformations) performed at 35° C. over a frequency range of from 2×10⁻² to 20 Hz on a rheometer (for example a Haake RS50 and RS75 rheometer) under a torsional/shear stress, for example in cone-plate geometry (for example with a cone angle of 1°).

When the materials are too cohesive for a person skilled in the art to be able to characterize them with the above Haake RS50 or RS75 rheometer (for example when G′_{AS}(2×10⁻² Hz, 35° C.)>10⁸ Pa), the viscoelastic properties of the materials are measured during dynamic tests under sinusoidal stresses of low amplitude (small deformations) performed at 35° C. over a frequency range of from 2×10⁻² to 20 Hz on a viscoelasticimeter, for example, a viscoelasticimeter of the type DMA 2980 from T.A. Instruments, in traction on film. To do this, the materials are dissolved in a suitable volatile solvent, for example, at a concentration of 10%, and then poured in the form of a film into a Teflon-coated matrix; they are then dried at 35° C. in order for the volatile solvent to evaporate off and for a film formed from these materials to be recovered. In this case, the elastic tensile modulus E′ and the viscous tensile modulus E″ are determined and the values G′ and G″ are calculated by means of the relationships G′=E′/3 and G″=E″/3.

The at least one pro-adhesive material as disclosed herein is chosen from, for example, block and random polymers and copolymers, chosen from:

polyurethanes,

acrylic polymers and copolymers,

silicones,

butyl rubbers, such as polyisobutylenes,

ethylene copolymers, such as ethylene/vinyl acetate, ethylene/esters and ethylene/olefins,

polyamides and polyamides optionally modified with fatty chains,

natural rubbers,

polyvinyl alcohols,

block and random copolymers comprising at least one monomeric residue, including, of course, a combination of monomeric residues. such copolymers having a glass transition temperature below room temperature (25° C.): the at least one monomeric residue can result from monomers chosen from butadiene, ethylene, propylene, isoprene, isobutylene, silicones, acrylic compounds, and methacrylic compounds, and blends thereof, and, of course,

mixtures thereof.

The glass transition temperature is, for example, the temperature that may be found in a handbook, for instance the Handbook of Polymers, 3rd edition, 1989, and that may be calculated by any known means in the art.

The at least one pro-adhesive material disclosed herein may be chosen, for example, from adhesives of “pressure-sensitive adhesive” type such as those mentioned in the “Handbook of Pressure Sensitive Adhesive Technology” 3rd Edition, D. Satas, the relevant disclosure of which is specifically incorporated by reference herein.

In one embodiment, the at least one pro-adhesive material is chosen from polyisobutylenes with a relative molar mass Mv of greater than or equal to 150 000, such as greater than or equal to 200 000, further such as greater than or equal to 300 000, and further still such as greater than or equal to 600 000.

This relative molar mass Mv may be evaluated by its viscosimetric mean, calculated according to the formula J₀=3.06×10⁻² Mv^{0.65}, in which J₀ is the Staudinger index (in cm³/g). This index is calculated from the flow time of a solution with a polyisobutylene concentration of C=0.01 g/cm³ in isooctane, through a capillary I of an Ubbelhode viscometer at 20° C. according to ISO standard 1628.

The relative molar mass Mv may also be evaluated by GPC (gel permeation chromatography) according to the following protocol (the molar mass by weight of polystyrene equivalents): 200 μl of a 0.5% solution of polymer (adhesive material) are injected by means of a Waters 6000 A pump, the eluent being a 100% THF solution, flow rate 1 ml/min, at room temperature, through a set of 8 columns: pstyragel 500 Å+10⁴ Å+2×10³ Å+styragel HR0.5+2×HR1+HR5E (300×7.8 mm). The detection is performed on a Waters 410 refractometer and on a Waters 490 UV detector at a wavelength of 254 nm. A pro-adhesive material is considered to have the Mv recited in paragraph [034] if such values are obtained by one or both of the viscosimetric mean and GPC methods described in paragraphs [035] and [036] herein.

Examples of commercial products suitable for the disclosed composition include polyisobutylenes with relative molar masses Mv greater than 150 000 of the Oppanol range from BASF, such as the polyisobutylenes with respective relative molar masses Mv of 200 000, 400 000 and 1 110 000 sold under the respective trade names “Oppanol B 30 SF”, “Oppanol B 50 SF” and “Oppanol B 100” by the company BASF, the polyisobutylenes with relative molar masses Mv ranging from 900 000 to 2 200 000 sold under the trade name “Vistanex MM” by the company Exxon, and blends thereof.

The at least one pro-adhesive material may, for example, be present in the composition disclosed herein in a content ranging from 0.1% to 99%, such as from 0.1% to 30% and further such as from 0.1% to 10% by weight, relative to the total weight of the composition.

The at least one diffusing compound present in the composition disclosed herein may be chosen from cosmetic oils and solvents that are not volatile at room temperature (about 25° C.), as a function of the chemical nature of the pro-adhesive material and the adhesive properties of the pro-adhesive material/diffusing compound combination.

Among the pro-adhesives defined above, a person skilled in the art may be led to make a selection of the pro-adhesive material/diffusing compound couple, wherein the pro-adhesive material can be chosen from those mentioned above, and the diffusing compound can be chosen from the oils and solvents conventionally used in cosmetics, such as those used in makeup.

This selection of the diffusing compound is made according to the criteria detailed below:

After interaction, the pro-adhesive material/diffusing compound couple forms a material known as an “adhesive system”. The diffusing compound develops the bonding power of the pro-adhesive material by diffusing into the polymer network of said material, during or after use on, for example, the skin, to obtain the desired cosmetic effect. It is important for the pro-adhesive material and the diffusing compound not to be in contact before use, by virtue of the presence of the medium defined above, so as not to give rise to at lease one effect chosen from a sticky feel and a reduction in the development of the bonding power during application and drying. In the case of a pulverulent composition, i.e., a composition comprising a pulverulent compound in a proportion from, for example, 60% to 99.9%, such as from 80% to 99.9% by weight, relative to the total weight of the composition, there is also a risk of instability of the composition, i.e., the powders can run a serious risk of becoming aggregated during contact between the pro-adhesive material and the diffusing compound.

The diffusion of the diffusing compound into the pro-adhesive material may be facilitated by using a volatile solvent in which said diffusing compound is conveyed.

In one embodiment, the “adhesive system” as disclosed herein satisfies the following condition:

G″_{AS}/G′_{AS}(0.2 Hz, 35° C.)≧0.35,

in which:

G″_{AS}(0.2 Hz, 35° C.) is the viscous shear modulus of said adhesive system, measured at a frequency of 0.2 Hz and at a temperature of 35° C.,

G′_{AS}(0.2 Hz, 35° C.) is the elastic shear modulus of said adhesive system, measured at a frequency of 0.2 Hz and at a temperature of 35° C.

In another embodiment, which may or may not be different from the previous embodiment, the “adhesive system” satisfies the following condition:

G′_{AS}(2 Hz, 35° C.)≧10⁴ Pa, wherein G′_{AS}(2 Hz, 35° C.) is the viscous shear modulus of said adhesive system, measured at a frequency of 2 Hz and at a temperature of 35° C.

In yet another embodiment, which may or may not be different from the previous embodiments, the “adhesive system” satisfies the following condition:

G′_{AS}(²×10⁻² Hz, 35° C.)≦5×10⁴ Pa, wherein G′_{AS}(2×10⁻² Hz, 35° C.) is the viscous shear modulus of said adhesive system, measured at a frequency of 2×10⁻² Hz and at a temperature of 35° C.

Further in another embodiment, the “adhesive system” satisfies the following three conditions:

G′_{AS}(2 Hz, 35° C.)≧10⁴ Pa,

G′_{AS}(2×10⁻² Hz, 35° C.)≦5×10⁴ Pa, and

G″_{AS}/G′_{AS}(0.2 Hz, 35° C.)≧0.35.

The at least one pro-adhesive material may, for example, be present in the composition disclosed herein in a content ranging from 0.1% to 99%, such as from 0.1% to 30% and further such as from 0.1% to 10% by weight, relative to the total weight of the composition.

The at least one diffusing compound may be chosen oils of low molecular mass, for example, with a molecular mass of less than or equal to 100 000 and such as less than or equal to 20 000. This at least one diffusing compound may be chosen from polyisobutylenes, lanolins, aliphatic and aromatic hydrocarbon-based resins, terpenic resins and mixtures thereof, resins of aliphatic and aromatic hydrocarbon-based “tackifying resin” type, as defined in the “Handbook of Pressure Sensitive Adhesive Technology”, D. Satas, 3rd Edition, p. 609. The at least one diffusing compound is at least partially, or even totally, compatible with the at least one pro-adhesive material. The term “compatible” means that there is a range of proportions in the mixture between the diffusing compound and the pro-adhesive material, such that an absence of macroscopic phase separation is visually observed.

Diffusing compounds that may be suitable for the disclosed composition include, for example, polyisobutylenes with a relative molar mass of less than 150 000, such as, the polyisobutylenes with respective relative molar masses Mv of 40 000, 55 000 and 85 000 sold under the respective trade names “Oppanol B 10”, “Oppanol B 12” and “Oppanol B 15” by the company BASF, and combinations thereof, with oils with a lower relative molar mass, such as polyisobutylenes with a relative molar mass Mv of less than or equal to 10 000, such as the polyisobutylenes of a relative molar mass ranging from 455 to 2000 sold under the trade name “Napvis” by the company BP Chemicals, the polyisobutylenes sold under the name “Parleam” by the Ets B. Rossow et Cie, and the polyisobutylenes sold under the trade name “Vistanex LM” by the company Exxon, and mixtures thereof.

The at least one diffusing compounds that may be used in combination with the at least one pro-adhesive material includes, for example, polyisobutylenes with relative molar masses greater than 150 000 of the Oppanol range from BASF, such as the polyisobutylenes with relative molar masses Mv of 200 000, 400 000 and 1 110 000 sold under the respective trade names “Oppanol B 30 SF”, “Oppanol B 50 SF” and “Oppanol B 100” by the company BASF, ande polyisobutylenes with relative molar masses (viscosimetric mean) ranging from 900 000 to 2 200 000, sold under the trade name “Vistanex MM” by the company Exxon, and mixtures thereof.

In one embodiment, the weight ratio between the at least one pro-adhesive material and the at least one diffusing compound ranges from 0.01:1 to 10:1, such as from 0.05:1 to 2:1, and further such as from 0.1:1 to 2:1.

Other “pro-adhesive material+diffusing compound” combinations that may be used for the disclosed composition include, for example, the pro-adhesive materials such as block polymers of the styrene-butadiene-styrene, styrene-(ethylene-butylene)-styrene or styrene-isoprene-styrene type, such as those sold under the trade name “Kraton” from Shell Chemical Co. and “Vector” from Exxon, combined with a resin chosen from aliphatic and aromatic hydrocarbon-based tackifying resins, such as the resins sold under the name “Piccotac” and “Hercotac” from Hercules and “Escorez” from Exxon, which may further be combined with at least one solvent.

Besides the pro-adhesive material and the diffusing compound defined above, the composition disclosed herein may further comprise at least one cosmetically acceptable support. The expression “cosmetically acceptable support” means a medium that is compatible with any keratin material such as the skin, the nails, the hair, the eyelashes and eyebrows, the mucous membranes and semi-mucous membranes and any other area of body and facial skin, and also any substitute for these supports, such as false nails, false eyelashes, and hairpieces.

This support may comprise any cosmetically acceptable compound chosen from water-soluble, water-dispersible, liposoluble and lipodispersible compounds conventionally used in cosmetics. Thus, the support may be in the form of a pulverulent composition, an anhydrous fatty phase (gel or solution), an aqueous phase (gel or solution), a dispersion, a solution in an organic solvent, an O/W or W/O emulsion, or a multiple emulsion, optionally stabilized with at least one organized system.

Depending on its nature, the at least one pro-adhesive material may be present in the support in a form chosen from a dissolved form and a dispersed form, and in a phase chosen from an aqueous phase and an anhydrous phase. The at least one pro-adhesive material may thus be in a form chosen from, for example, an aqueous dispersion of particles and a form of an oily or organic, i.e. non-aqueous, dispersion of particles.

The at least one pro-adhesive material and the at least one diffusing compound should be separated in the composition disclosed herein by at least one medium capable of limiting the molecular diffusions between the at least one pro-adhesive material and the at least one diffusing compound within the composition, and capable of allowing the diffusions during application or after application.

Depending on both the nature of the pro-adhesive material and of the diffusing compound, and also on the choice of the continuous medium made by a person skilled in the art, such as an aqueous continuous medium as compared to an anhydrous medium, various media capable of limiting the molecular diffusions between the pro-adhesive material and the diffusing compound within the composition, and allowing these diffusions during application or after application, may, for example, be envisaged, as described below.

In the case of a pulverulent composition, the continuous medium of the composition may be likened to that of the binder present in said composition.

Case in Which the Diffusing Compound is Not Present in the Continuous Medium

In this first embodiment, the at least one diffusing compound should be separated from the continuous medium in order to prevent it from diffusing into the at least one pro-adhesive material also contained in the composition. Four types of medium below capable of limiting the molecular diffusions between the pro-adhesive material and the diffusing compound within the composition, and allowing these diffusions during application or after application, can, for example, be as follows:

1. Said medium may be in a form chosen from a monolayer and a multilayer of surfactants, monomers and polymers, a portion of which is aliphatic and solid at room temperature. The term “solid portion” means at least partially crystallized, such that a melting peak is observed in DSC (Differential Scanning Calorimetry), at a temperature above room temperature.

In one embodiment, the diffusing compound is lipophilic and is placed in an oil/water emulsion with at least one surfactant whose fatty chains are unbranched and saturated, with a chain length of greater than 14 carbon atoms such as 16 carbon atoms per molecule. Such surfactant is chosen, for example, from linear and saturated fatty alcohols comprising at least 16 carbon atoms per molecule and ethoxylated, and fatty acids comprising at least 16 carbon atoms per molecule and neutralized with a base such as triethanolamine.

2. Said medium may be in the form of a polymer shell, and optionally of a surfactant, thus forming nanocapsules such as those described and used in European Patent No. 0 447 318 A1.

3. Said medium may be in the form of a stack of at least two lipid bilayers, thus forming multilamellar vesicles, i.e., liposomes, such as those described and used in French Patent No. 2 315 991.

In another embodiment, the diffusing compound is hydrophilic and dissolved in the inner phase of the liposomes. In this case, a restriction may be placed on the nature of the diffusing compound, which should not become incorporated into the lamellar phases of the lipid bilayers.

Further in another embodiment, the diffusing compound is lipophilic and present in the inner phase of multilamellar vesicles, the outer phase of the vesicles being aqueous. These are then referred to as oleosomes, as defined in European Patent No. 0 641 557.

4. Said medium may be in the form of a liquid medium and of two interfaces of monolayers of amphiphilic molecules; these are then referred to, for example, as W/O/W or O/W/O triple emulsions, such as those described or mentioned in French Patent No. 2 756 178.

Depending on the case, a person skilled in the art can select the vehicle that is best suited for the pro-adhesive material. Said pro-adhesive material may, for example, be present in the composition in a dissolved form, which will then be emulsified, or in the continuous medium, or in dispersed form (such as in polymer form in an aqueous continuous medium commonly known as a latex, and in the form of a dispersion of polymer particles in a non-aqueous medium, such as those described in patent application WO 97/00662 in the case of a lipophilic medium).

The diffusing compound may be placed in contact with any constituent usually used in cosmetics, provided that a person skilled in the art can manage formulation.

Case in Which the Diffusing Compound is Present in the Continuous Medium

In a second embodiment, the diffusing compound can be conveyed in the continuous medium. The pro-adhesive material should be separated from the continuous medium in order to prevent it from diffusing into the diffusing compound, which is also contained in the composition. Four types of medium below capable of limiting the molecular diffusions between the pro-adhesive material and the diffusing compound within the composition, and capable of allowing these diffusions during application or after application, can be used, in the same way as in the first embodiment above:

1. Pro-adhesive material encapsulated in nanocapsules;

2. Pro-adhesive material encapsulated in liposomes or oleosomes;

3. Pro-adhesive material in the inner phase of a W/O/W or O/W/O triple emulsion;

4. Pro-adhesive material present in a dispersed form inside the droplets of dispersed phase of an emulsion.

In one embodiment, the pro-adhesive material is in a form chosen from the latex form and the form of a dispersion in anhydrous medium (NAD).

The pro-adhesive material may be placed in contact with any constituent usually used in cosmetics, provided that a person skilled in the art can manage to formulate.

When the composition disclosed herein comprises a fatty phase, it can, for example, comprise at least one oil chosen from cosmetically acceptable oils and physiologically acceptable oils chosen, for example, from at least one of carbon-based oils, hydrocarbon-based oils and silicone oils of mineral, animal, plant and synthetic origins, provided that they are compatible with the intended use.

Examples of the hydrocarbon-based oils that may be used include liquid paraffin, liquid petroleum jelly, mink oil, turtle oil, soybean oil, perhydrosqualene, sweet almond oil, beauty-leaf oil, palm oil, grapeseed oil, sesame seed oil, corn oil, parleam oil, arara oil, rapeseed oil, sunflower oil, cottonseed oil, apricot oil, castor oil, avocado oil, jojoba oil, olive oil and cereal germ oil; esters of lanolic acid, of oleic acid, of lauric acid and of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyidodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid and isostearic acid; higher fatty alcohols such as cetanol, stearyl alcohol and oleyl alcohol, linoleyl alcohol and linolenyl alcohol, isostearyl alcohol and octyldodecanol; silicone oils such as polydimethylsiloxanes (PDMS), which are optionally phenylated such as phenyltrimethicones, and optionally substituted with at least one group chosen from aliphatic groups, aromatic groups, and functional groups such as hydroxyl, thiol, and amine groups; and polysiloxanes modified with at least one entity chosen from fatty acids, fatty alcohols and polyoxyalkylenes, and mixtures thereof.

For example, the at least one oil that is volatile at room temperature may be used. The term “volatile oil” means an oil that is capable of evaporating from the skin at room temperature in less than one hour. For example, the volatile oil has a viscosity ranging from 0.5 to 25 centistokes at 25° C. Oils whose flash point is high enough to allow these oils to be used in the composition may also be used. These volatile oils can also facilitate the application of the composition to the skin.

These oils may be chosen from hydrocarbon-based oils and silicone oils optionally comprising at least one group chosen from alkyl and alkoxy groups, at the end of a silicone chain or pendent thereon.

Volatile silicone oils that may be used include, for example, silicones chosen from linear and cyclic silicones comprising from 2 to 7 silicon atoms, optionally comprising at least one group chosen from alkyl and alkoxy groups comprising from 1 to 10 carbon atoms. For example, octamethylcyclotetrasiloxane, decamethylcyclopentasiloxane, hexadecamethylcyclohexasiloxane, heptamethylhexyltrisiloxane and heptamethyloctyltrisiloxane, and mixtures thereof may be used.

Volatile hydrocarbon-based oils that may be used include, for example, C₈-C₁₆ isoparaffins such as isododecane, isodecane, heptane and isohexadecane, and mixtures thereof.

The at least one volatile oil may be present in the composition in a content ranging from 0.1% to 99% by weight, such as from 0.1% to 60% and further such as from 0.1% to 40%, by weight relative to the total weight of the composition.

The fatty phase may also comprise at least one component chosen from waxes, gums and pasty fatty substances, of plant, animal, mineral and synthetic origin, and even silicone-based.

Examples of the waxes that are solid at room temperature which may be used in the composition include hydrocarbon-based waxes such as beeswax, carnauba wax, candelilla wax, ouricury wax, Japan wax, cork fibre wax and sugar cane wax, paraffin wax, lignite wax, microcrystalline waxes, lanolin wax, montan wax, ozokerites, polyethylene waxes, the waxes obtained by Fischer-Tropsch synthesis, hydrogenated oils, fatty esters and glycerides that are solid at 25° C. Silicone waxes may also be used, including, for example, alkyl, alkoxy and/or esters of polymethylsiloxane. The waxes may be in the form of stable dispersions of colloidal wax particles as may be prepared according to known methods, such as those of “Microemulsions Theory and Practice”, L. M. Prince Ed., Academic Press (1977), pages 21-32. Mention may be made of jojoba oil as a wax that is liquid at room temperature.

The waxes may be present in a proportion ranging, for example, from 0.1% to 50% by weight relative to the total weight of the composition.

The pasty fatty substances may be defined by means of at least one of the following physicochemical properties:

a viscosity ranging from 0.1 to 40 Pa.s (1 to 400 poises) and such as from 0.5 to 25 Pa.s, measured at 40° C. with a Contraves TV rotary viscometer equipped with an MS-r3 or MS-r4 rotor at a frequency of 240 rpm, and

a melting point of 25-70° C., such as 25-55° C.

The compositions disclosed herein may also comprise at least one component chosen from alkyl, alkoxy and phenyl dimethicones, such as the product sold under the name “Abil Wax 2440” by the company Goldschmidt.

The fatty phase may also comprise at least one liposoluble dye. The at least one liposoluble dye may be chosen from, for example, Sudan red, DC Red 17, DC Green 6, β-carotene, soybean oil, Sudan brown, DC Yellow 11, DC Violet 2, DC Orange 5,and quinoline yellow. The at least one dye may represent, for example, from 0.01 to 20% by weight of the total weight of the composition, such as from 0.1% to 6% by weight of the total weight of the composition.

The fatty phase can represent, for example, from 0.01% to 99.9% by weight relative to the total weight of the composition. When the composition disclosed herein is a powder, for example for the face, the fatty phase is, for example, present in a content ranging from 0.1% to 30% by weight relative to the total weight of the composition.

The composition disclosed herein may further comprise at least one organic solvent, such as when the composition is a nail varnish.

The at least one organic solvent that may be used includes, for example:

ketones that are liquid at room temperature, such as methyl ethyl ketone, methyl isobutyl ketone, diisobutyl ketone, isophorone, cyclohexanone and acetone;

alcohols that are liquid at room temperature, such as ethanol, isopropanol, diacetone alcohol, 2-butoxyethanol and cyclohexanol;

glycols that are liquid at room temperature, such as ethylene glycol, propylene glycol, pentylene glycol and glycerol;

propylene glycol ethers that are liquid at room temperature, such as propylene glycol monomethyl ether, propylene glycol monomethyl ether acetate and dipropylene glycol mono-n-butyl ether;

short-chain esters (comprising from 3 to 8 carbon atoms in total), such as ethyl acetate, methyl acetate, propyl acetate, n-butyl acetate and isopentyl acetate;

ethers that are liquid at room temperature, such as diethyl ether, dimethyl ether and dichlorodiethyl ether;

alkanes that are liquid at room temperature, such as decane, heptane, dodecane and cyclohexane;

cycloaromatic compounds that are liquid at room temperature, such as toluene and xylene; and

aldehydes that are liquid at room temperature, such as benzaldehyde and acetaldehyde, and

mixtures thereof.

The at least one organic solvent may be present in the composition in a content ranging, for example, from 0.01% to 99% by weight, such as from 50% to 99% by weight, for use as a nail varnish, relative to the total weight of the composition.

When the composition disclosed herein comprises an aqueous phase, it may comprise at least one component chosen from water, floral water such as cornflower water, mineral water such as eau de Vittel, eau de Lucas and eau de La Roche Posay, and spring water.

The aqueous phase may also comprise at least one solvent other than water, such as primary alcohols, for example, ethanol and isopropanol, glycols such as propylene glycol, butylene glycol, dipropylene glycol and diethylene glycol, glycol ethers such as mono-, di- or tripropylene glycol (C₁-C₄)alkyl ethers, and mono-, di- or triethylene glycol, and mixtures thereof.

The aqueous phase may also comprise at least one water-soluble colorant chosen from the colorants that are common in the field under consideration, such as the disodium salt of ponceau, the disodium salt of alizarin green, quinoline yellow, the trisodium salt of amaranth, the disodium salt of tartrazine, the monosodium salt of rhodamine, the disodium salt of fuchsin, and xanthophylls, and mixtures thereof.

The aqueous phase may also comprise any compound that is compatible with an aqueous phase, i.e., water-soluble or water-dispersible, such as gelling agents, film-forming polymers, thickeners, surfactants, preserving agents and pigment dispersions.

When the composition as disclosed herein is a nail varnish, the aqueous phase may comprise water and at least one aqueous-alcoholic mixture comprising, for example, at least one component chosen from C₁-C₅ monoalcohols and C₂-C₈ glycols.

For example, the aqueous phase is present in the disclosed composition in a content ranging from 0.01% to 99% by weight, such as from 0.1% to 80% by weight, relative to the total weight of the composition. When the composition is a powder, for example, for the face, the aqueous phase is, for example, present in a content ranging from 0.01% to 30% by weight relative to the total weight of the composition.

The composition disclosed herein may also comprise at least one amphiphilic compound, i.e. a compound comprising both a lipophilic portion (apolar portion) and a hydrophilic portion (polar portion), which may be adsorbed onto a surface or an interface. Such compounds can be chosen from, for example, emulsifiers and co-emulsifiers.

The emulsifiers and co-emulsifiers used in the composition, when the composition is in an emulsion form, are chosen from those conventionally used in the fields of cosmetics and dermatology. The emulsifier and the co-emulsifier may be present in the composition in a proportion ranging, for example, from 0.3% to 30% by weight, such as from 0.5% to 20% by weight, relative to the total weight of the composition.

O/W surfactants that may be used, for example, include (CTFA): cetearylglucoside, PEG-40 stearate, sorbitan tristearate, sorbitan stearate, polysorbate 60, sorbitan stearate/sucrose cocoate mixture, glyceryl stearate/PEG-100 stearate mixture, PEG-400, glyceryl stearate, and PEG-6/PEG-32/glycol stearate mixture. W/O surfactants that may be used also include, for example, the polyglyceryl-4 isostearate/cetyldimethicone copolyol/hexyl laurate mixture and the mineral oil/petrolatum/ozokerite/glyceryl oleate/lanolin alcohol mixture.

The composition disclosed herein may also additionally comprise at least one pulverulent compound. The at least one pulverulent compound may be chosen from pigments, nacres, and fillers usually used in cosmetic compositions. For example, the at least one pulverulent compound represents from 0.1% to 99.9%, such as from 60% to 99.9% by weight, relative to the total weight of said composition. When said composition is a powder, for example for the face, the at least one pulverulent compound may, for example, represent from 70% to 99.9% by weight, relative to the total weight of the composition.

The pigments may be chosen from white and coloured, and mineral and organic pigments. The mineral pigments that may be used include, for example, titanium dioxide, optionally surface-treated, zirconium oxide, cerium oxide, iron oxide, chromium oxide, manganese violet, ultramarine blue, chromium hydrate and ferric blue. The organic pigments that may be used include, for example, carbon black, pigments of D & C type, and lakes based on cochineal carmine, barium, strontium, calcium and aluminium.

The nacreous pigments may be chosen from, for example, white nacreous pigments such as mica coated with a metal chosen from titanium and bismuth oxychloride, coloured nacreous pigments such as titanium mica with iron oxides, titanium mica with, for example, ferric blue and chromium oxide, titanium mica with an organic pigment of the abovementioned type, and also nacreous pigments based on bismuth oxychloride.

The fillers may be chosen from mineral and organic, lamellar and spherical fillers, such as talc, mica, silica, kaolin, Nylon powders (Orgasol from Atochem or Nylon 12), poly-β-alanine powders and polyethylene powders, Teflon, bismuth oxychloride, lauroyllysine, starch, boron nitride, tetrafluoroethylene polymer powders, hollow microspheres such as Expancel (Nobel Industrie), Polytrap (Dow Corning) and silicone resin microbeads (for example Tospearls from Toshiba), precipitated calcium carbonate, magnesium carbonate, magnesium hydrocarbonate, hydroxyapatite, hollow silica microspheres (Silica Beads from Maprecos), glass or ceramic microcapsules, metal soaps derived from organic carboxylic acids comprising from 8 to 22 carbon atoms, such as from 12 to 18 carbon atoms, for example, zinc, magnesium, and lithium stearate, zinc laurate and magnesium myristate.

The composition may also comprise any compound usually used in such compositions, such as thickeners, antioxidants, fragrances, preserving agents, surfactants and active agents.

Cosmetic, dermatological or hygiene active agents that may be used in the composition disclosed herein include, for example, moisturizers, vitamins, essential fatty acids, sphingolipids and sunscreens. These active agents are present in an amount generally used in powders. For example, they are present in a proportion ranging from 0.001 to 20% by weight relative to the total weight of the composition.

A person skilled in the art will take care to select this or these optional additional compound(s), and/or the amount thereof, such that the advantageous properties of the composition disclosed herein are not, or are not substantially, adversely affected by the envisaged addition.

The compositions disclosed herein may be prepared in the usual manner by those skilled in the art. They may be in the form of a free or compact powder, an anhydrous gel, an aqueous gel, a W/O or O/W emulsion or a multiple emulsion. They may be, for example, in the form of a stick or dish that may be used by taking up with a finger or with a sponge, or in the form of cream: for example, they can be used as blushers, face powders, eyeshadows, lip powders, care or makeup powders for the face or the body, deodorant powders, lipsticks, foundations, mascaras, eyeliners and concealer products. They may also be in the form of a free powder, for example, as face powders, care or makeup powders for the face or the body, and deodorant powders. They may also be in the form of a solution or a dispersion and may be used as nail varnishes.

These compositions for topical application may, for example, constitute a cosmetic composition, a protective hygiene composition, a medicated composition and a care composition for the face, for the neck, for the hands, for the integuments and for the body, such as a fragranced or unfragranced anhydrous care powder, and such as antisun powder, a makeup composition such as a makeup gel, and an artificial tanning composition.

The compositions disclosed herein may be used alone, in application to a surface chosen from body skin, facial skin and mucous membranes. These compositions can have enhanced staying power and improved transfer-resistance properties once applied.

The invention is illustrated in greater detail in the examples that follow.

### EXAMPLE 1

### Foundation

Oil-in-water emulsion foundation, in which the pro-adhesive material and the diffusing compound are both emulsified separately in water, and the medium capable of limiting the molecular diffusions between the pro-adhesive material and the diffusing compound within the formulation and capable of allowing these diffusions during application consists of a layer of surfactant (triethanolamine stearate), wherein said surfactant is derived from the neutralization of stearic acid, the melting point of which is about 60° C., with a base, triethanolamine:

A first emulsion, noted E1, was prepared, wherein the emulsion is composed of:

| | |
|---|---|
| Oppanol B100 polyisobutylene | 6% |
| Isododecane | 60% |
| Oleic acid | 1.1% |
| Triethanolamine | 1.6% |
| Preserving agents | 0.2% |
| qs water | |

A second emulsion, noted E2, was prepared, wherein the emulsion is composed of:

| | |
|---|---|
| Napvis D07 polyisobutylene | 24% |
| Stearic acid | 2.66% |
| Triethanolamine | 1.33% |
| Pigments (black, brown, etc. iron oxide) | 14% |
| Nylon powder (Orgasol) | 8% |
| Preserving agents | 0.2% |
| Water | qs 100% |

These emulsions were prepared according to the standard methods for preparing emulsions.

Emulsions E1 and E2 were then mixed together in a 50/50 ratio (by mass).

The adhesive system consisting of the pro-adhesive material of emulsion E1 and of the diffusing compound of emulsion E2 in a 1/4 ratio has the following characteristics:

G′_{AS}(2 Hz, 35° C.)=7×10³ Pa,

G′_{AS}(2×10⁻² Hz, 35° C.)=2×10³ Pa, and

G″_{AS}/G′_{AS}(0.2 Hz, 35° C.)=0.35

The composition thus obtained was applied, for example, to a person's face.

The mechanical and physical stresses generated during the application result in rupture of the layers of surfactants that surrounded the emulsion globules that contained the diffusing compound and the pro-adhesive material, respectively, thus allowing them to interact.

The makeup product thus obtained has enhanced staying power and shows very little transfer.

### EXAMPLE 2

### Makeup Powder

A binder of oil-in-water emulsion type was first prepared, wherein in the binder, the pro-adhesive material and the diffusing compound are both emulsified separately in water, and the medium capable of limiting the molecular diffusions between the pro-adhesive material and the diffusing compound within the formulation and capable of allowing these diffusions during application consists of a layer of surfactant (triethanolamine stearate), wherein said surfactant is derived from the neutralization of the stearic acid, the melting point of which is about 60° C., with a base, triethanolamine:

A first emulsion, noted E1, was prepared, wherein the emulsion is composed of:

| | |
|---|---|
| Oppanol B100 polyisobutylene | 6% |
| Isododecane | 60% |
| Oleic acid | 1.1% |
| Triethanolamine | 1.6% |
| Preserving agents | 0.2% |
| qs water | |

A second emulsion, noted E2, was prepared, wherein the emulsion is composed of:

| | |
|---|---|
| Napvis D07 polyisobutylene | 48% |
| Stearic acid | 5.32% |
| Triethanolamine | 2.66% |
| Preserving agents | 0.2% |
| Water | qs 100% |

These emulsions were prepared according to the standard methods for preparing, emulsions.

Emulsions E1 and E2 were then mixed together in a 2/1 ratio (by mass) to obtain the binder.

The adhesive system consisting of the pro-adhesive material of emulsion E1 and of the diffusing compound of emulsion E2 in a 1/4 ratio has the following characteristics:

G′_{AS}(2 Hz, 35° C.)=7×10³ Pa,

G′_{AS}(2×10⁻² Hz, 35° C.)=2×10⁻³ Pa, and

G″_{AS}/G′_{AS}(0.2 Hz, 35° C.)=0.35

The makeup powder consists of:

| | |
|---|---|
| Binder | 20% |
| Pulverulent phase: | |
| Talc | 58.9% |
| Iron oxide | 2.8% |
| Nylon powder | 10% |
| Titanium oxide | 3% |
| Methylsilsequioxane resin | 5% |
| Preserving agents | 0.3% |

The powder was obtained in the manner described below. The compounds of the pulverulent phase were mixed together in a turbomixer. This mixture was then ground using a toothed roll crusher or an air-jet mill. Lastly, the binder was added dropwise to the pulverulent phase with stirring in a turbomixer whose temperature was kept constant at 20° C. The two phases were mixed together until a homogeneous product was obtained.

Since the pro-adhesive material and the diffusing compound were separated in the composition, a pulverulent form was indeed obtained without aggregates. This is an advantageous property of the invention in the case of pulverulent forms.

The composition thus obtained was applied, for example, to a person's face.

The mechanical and physical stresses generated during the application result in rupture of the layers of surfactants that surrounded the emulsion globules that contained the diffusing compound and the pro-adhesive material, respectively, thus allowing them to interact.

The makeup product thus obtained has enhanced staying power and shows very little transfer.

What is claimed is:

1. A composition chosen from cosmetic and physiological compositions comprising:
i) at least one pro-adhesive material that satisfies the following condition, for any frequency ranging from 2×10⁻² to 2 Hz: 10⁸ Pa≧G′(35° C.)≧2×10⁴ Pa,
wherein:
G′(35° C.) is the elastic shear modulus of said at least one pro-adhesive material, measured at a temperature of 35° C.;
ii) at least one diffusing compound,
wherein a system created by the interaction of the at least one pro-adhesive material and the at least one diffusing compound is an adhesive system that satisfies the following conditions:
G′_{AS}(2 Hz, 35° C.)≧10³ Pa, and
G′_{AS}(2×10⁻² Hz, 35° C.)≦3×10⁵ Pa,
wherein:
G′_{AS}(2 Hz, 35° C.) is the elastic shear modulus of the adhesive system, measured at a frequency of 2 Hz and at a temperature of 35° C., and
G′_{AS}(2×10⁻² Hz, 35° C.) is the elastic shear modulus of the adhesive system, measured at a frequency of 2×10⁻² Hz and at a temperature of 35° C.; and
iii) at least one medium for separating the at least one pro-adhesive material and the at least one diffusing compound, wherein the at least one medium limits the molecular diffusion between the at least one pro-adhesive material and the at least one diffusing compound within the composition, and wherein the weight ratio between the at least one pro-adhesive material and the at least one diffusing compound ranges from 0.01:1 to 10:1.

2. The composition according to claim 1, wherein the at least one pro-adhesive material satisfies the following condition, for any frequency ranging from 2×10⁻² to 2 Hz:
10⁸ Pa≧G′(35° C.)≧10⁵ Pa.

3. The composition according to claim 2, wherein the at least one pro-adhesive material satisfies the following condition, for any frequency ranging from 2×10⁻² to 2 Hz:
5×10⁷ Pa≧G′(35° C.)≧3×10⁵ Pa.

4. The composition according to claim 3, wherein the at least one pro-adhesive material satisfies the following condition, for any frequency ranging from 2×10⁻² to 2 Hz:
10⁷ Pa≧G′(35° C.)≧3×10⁵ Pa.

5. The composition according to claim 1, wherein the weight ratio between the at least one pro-adhesive material and the at least one diffusing compound ranges from 0.05:1 to 2:1.

6. The composition according to claim 5, wherein the weight ratio between the at least one pro-adhesive material and the at least one diffusing compound ranges from 0.1:1 to 2:1.

7. A composition chosen from cosmetic and physiological compositions for applying to a keratin material, comprising:
i) at least one pro-adhesive material that satisfies the following condition, for any frequency ranging from 2×10² to 2 Hz: 108 Pa≧G′(35° C.)≧2×10⁴ Pa,
wherein:
G′(35° C.) is the elastic shear modulus of said at least one pro-adhesive material, measured at a temperature of 35° C.;
ii) at least one diffusing compound,
wherein a system created by interaction of the at least one pro-adhesive material and the at least one diffusing compound is an adhesive system that satisfies the following conditions:
G′_{AS}(2 Hz, 35° C.)≧10³ Pa, and
G′_{AS}(2×10⁻² Hz, 35° C.)≦3×10 Pa,
wherein:
G′_{AS}(2 Hz, 35° C.) is the elastic shear modulus of the adhesive system, measured at a frequency of 2 Hz and at a temperature of 35° C., and
G′_{AS}(2×10⁻² Hz, 35° C.) is the elastic shear modulus of the adhesive system, measured at a frequency of 2×10⁻² Hz and at a temperature of 35° C.; and
iii) at least one medium for separating the at least one pro-adhesive material and the at least one diffusing compound, wherein the at least one medium limits the molecular diffusion between the at least one pro-adhesive material and the at least one diffusing compound within the composition, and wherein the weight ratio between the at least one pro-adhesive material and the at least one diffusing compound ranges from 0.01:1 to 10:1, further wherein, during application or after application, said interaction can take place.

8. The composition according to claim 7, wherein the at least one pro-adhesive material satisfies the following condition, for any frequency ranging from 2×10⁻² to 2 Hz:
10⁸ Pa≧G′(35° C.)≧10⁵ Pa.

9. The composition according to claim 8, wherein the at least one pro-adhesive material satisfies the following condition, for any frequency ranging from 2×10⁻² to 2 Hz:
5×10⁷ Pa≧G′(35° C.)≧3×10⁵ Pa.

10. The composition according to claim 9, wherein the at least one pro-adhesive material satisfies the following condition, for any frequency ranging from 2×10⁻² to 2 Hz:
10⁷ Pa≧G′(35° C.)≧3×10⁵ Pa.

11. The composition according to claim 7, wherein the weight ratio between the at least one pro-adhesive material and the at least one diffusing compound ranges from 0.05:1 to 2:1.

12. The composition according to claim 11, wherein the weight ratio between the at least one pro-adhesive material and the at least one diffusing compound ranges from 0.1:1 to 2:1.

13. The composition according to claim 1, wherein the adhesive system further satisfies the following condition:
G″_{AS}/G′_{AS}(0.2 Hz, 35° C.)≧0.35,
wherein:
G″_{AS}(2 Hz, 35° C.) is the viscous shear modulus of the adhesive system, measured at a frequency of 0.2 Hz and at a temperature of 35° C.,
G′_{AS}(0.2 Hz, 35° C.) is the elastic shear modulus of the adhesive system, measured at a frequency of 0.2 Hz and at a temperature of 35° C.

14. The composition according to claim 1, wherein:
G′_{AS}(2 Hz, 35° C.)≧10⁴ Pa.

15. The composition according to claim 1, wherein:
G′_{AS}(2×10⁻² Hz, 35° C.)≦5×10⁴ Pa.

16. The composition according to claim 13, wherein:
G′_{AS}(2 Hz, 35° C.)≧2×10⁴ Pa,
G′_{AS}(2×10⁻² Hz, 35° C.)≦5×10⁴ Pa, and
G″_{AS}/G′_{AS}(0.2 Hz, 35° C.)≧0.35.

17. The composition according to claim 1, wherein the at least one pro-adhesive material is chosen from:
polyurethanes,
acrylic polymers and copolymers,
silicones,
butyl rubbers,
ethylene copolymers,
polyamides optionally modified with fatty chains,
natural rubbers,
polyvinyl alcohols, and
blockand random copolymers comprising at least one monomeric residue has and having a glass transition temperature below room temperature (25° C.).

18. The composition according to claim 1, wherein the at least one pro-adhesive material is chosen from:
polyurethanes,
acrylic polymers and copolymers,
silicones,
polyisobutylenes,
ethylene/vinyl acetate, ethylene/ester and ethylene/olefin copolymers,
polyamides optionally modified with fatty chains,
natural rubbers, and
block and random copolymers comprising at least one monomeric residue and having a glass transition temperature below room temperature (25° C.), wherein said monomer is chosen from butadiene, ethylene, propylene, isoprene, isobutylene, silicones, acrylic compounds and methacrylic compounds.

19. The composition according to claim 1, wherein the at least one pro-adhesive material is chosen from polyisobutylenes with a relative molar mass Mv of greater than or equal to 150 000.

20. The composition according to claim 19, wherein the at least one pro-adhesive material is chosen from polyisobutylenes with a relative molar mass Mv of greater than or equal to 200 000.

21. The composition according to claim 20, wherein the at least one pro-adhesive material is chosen from polyisobutylenes with a relative molar mass Mv of greater than or equal to 300 000.

22. The composition according to claim 21, wherein the at least one pro-adhesive material is chosen from polyisobutylenes with a relative molar mass Mv of greater than or equal to 600 000.

23. The composition according to claim 1, wherein the at least one pro-adhesive material is present in a content ranging from 0.1% to 99% by weight, relative to the total weight of the composition.

24. The composition according to claim 23, wherein the at least one pro-adhesive material is present in a content ranging from 0.1% to 30% by weight, relative to the total weight of the composition.

25. The composition according to claim 24, wherein the at least one pro-adhesive material is present in a content ranging from 0.1% to 10% by weight, relative to the total weight of the composition.

26. The composition according to claim 1, wherein the at least one diffusing compound is chosen from cosmetic oils and solvents that are not volatile at room temperature.

27. The composition according to claim 1, wherein the at least one diffusing compound is chosen from oils of low molecular mass.

28. The composition according to claim 27, wherein the at least one diffusing compound is chosen from oils with a molecular mass of less than or equal to 100 000.

29. The composition according to claim 28, wherein the at least one diffusing compound is chosen from oils with a molecular mass of less than or equal to 20 000.

30. The composition according to claim 1, wherein the at least one diffusing compound is chosen from polyisobutylenes, lanolins, aliphatic and aromatic hydrocarbon-based resins, terpenic resins, and aliphatic and aromatic hydrocarbon-based tackifying resins.

31. The composition according to claim 30, wherein the at least one diffusing compound is chosen from polyisobutylenes with a relative molar mass of less than 150 000.

32. The composition according to claim 1, wherein the at least one pro-adhesive material is chosen from block polymers of styrene-butadiene-styrene, styrene-(ethylene-butylene)-styrene and styrene-isoprene-styrene type and the at least one diffusing compound is chosen from aliphatic and aromatic hydrocarbon-based tackifying resins.

33. The composition according to claim 1, wherein the at least one medium is chosen from monolayers and multilayers of amphiphilic molecules that are solid at room temperature, and layers of polymers.

34. The composition according to claim 1, wherein the at least one medium is chosen from:
monolayers and multilayers of monomer and polymer surfactants, a portion of which is aliphatic and solid at room temperature,
a polymer shell,
a stack of at least two lipid bilayers, and
a liquid medium and two interfaces of monolayers of amphiphilic molecules.

35. The composition according to claim 1, wherein the at least one pro-adhesive material is in a dispersed form.

36. The composition according to claim 1, further comprising at least one cosmetically acceptable support.

37. The composition according to claim 7, wherein the adhesive system further satisfies the following condition:
G″_{AS}/G′_{AS}(0.2 Hz, 35° C.)≧0.35,
wherein:
G″_{AS}(0.2 Hz, 35° C.) is the viscous shear modulus of the adhesive system, measured at a frequency of 0.2 Hz and at a temperature of 35° C.,
G′_{AS}(0.2 Hz, 35° C.) is the elastic shear modulus of the adhesive system, measured at a frequency of 0.2 Hz and at a temperature of 35° C.

38. The composition according to claim 7, wherein:
G′_{AS}(2 Hz, 35° C.)≧10⁴ Pa.

39. The composition according to claim 7, wherein:
G′_{AS}(2×10⁻² Hz, 35° C.)≦5×10⁴ Pa.

40. The composition according to claim 37, wherein:
G′_{AS}(2 Hz, 35° C.)≧10⁴ Pa,
G′_{AS}(2×10⁻² Hz, 35° C.)≦5×10⁴ Pa, and
G″_{AS}/G′AS(0.2 Hz, 35° C.)≧0.35.

41. The composition according to claim 7, wherein the at least one pro-adhesive material is chosen from:
polyurethanes,
acrylic polymers and copolymers,
silicones,
butyl rubbers,
ethylene copolymers,
polyamides optionally modified with fatty chains,
natural rubbers,
polyvinyl alcohols, and
blockand random copolymers comprising at least one monomeric residue and having a glass transition temperature below room temperature (25° C.).

42. The composition according to claim 7, wherein the at least one pro-adhesive material is chosen from:
polyurethanes,
acrylic polymers and copolymers,
silicones,
polyisobutylenes,
ethylene/vinyl acetate, ethylene/ester and ethylene/olefin copolymers,
polyamides optionally modified with fatty chains,
natural rubbers, and
block and random copolymers comprising at least one monomeric residue and having a glass transition temperature below room temperature (25° C.), wherein said monomer is chosen from butadiene, ethylene, propylene, isoprene, isobutylene, silicones, acrylic compounds and methacrylic compounds.

43. The composition according to claim 7, wherein the at least one pro-adhesive material is chosen from polyisobutylenes with a relative molar mass Mv of greater than or equal to 150 000.

44. The composition according to claim 43, wherein the at least one pro-adhesive material is chosen from polyisobutylenes with a relative molar mass Mv of greater than or equal to 200 000.

45. The composition according to claim 44, wherein the at least one pro-adhesive material is chosen from polyisobutylenes with a relative molar mass Mv of greater than or equal to 300 000.

46. The composition according to claim 45, wherein the at least one pro-adhesive material is chosen from polyisobutylenes with a relative molar mass Mv of greater than or equal to 600 000.

47. The composition according to claim 7, wherein the at least one pro-adhesive material is present in a content ranging from 0.1% to 99% by weight, relative to the total weight of the composition.

48. The composition according to claim 47, wherein the at least one pro-adhesive material is present in a content ranging from 0.1% to 30% by weight, relative to the total weight of the composition.

49. The composition according to claim 48, wherein the at least one pro-adhesive material is present in a content ranging from 0.1% to 100% by weight, relative to the total weight of the composition.

50. The composition according to claim 7, wherein the at least one diffusing compound is chosen from cosmetic oils and solvents that are not volatile at room temperature.

51. The composition according to claim 7, wherein the at least one diffusing compound is chosen from oils of low molecular mass.

52. The composition according to claim 51, wherein the at least one diffusing compound is chosen from oils with a molecular mass of less than or equal to 100 000.

53. The composition according to claim 52, wherein the at least one diffusing compound is chosen from oils with a molecular mass of less than or equal to 20 000.

54. The composition according to claim 7, wherein the at least one diffusing compound is chosen from polyisobutylenes, lanolins, aliphatic and aromatic hydrocarbon-based resins, terpenic resins, and aliphatic and aromatic hydrocarbon-based tackifying resins.

55. The composition according to claim 54, wherein the at least one diffusing compound is chosen from polyisobutylenes with a relative molar mass of less than 150 000.

56. The composition according to claim 7, wherein the at least one pro-adhesive material is chosen from block polymers of styrene-butadiene-styrene, styrene-(ethylene-butylene)-styrene and styrene-isoprene-styrene type and the at least one diffusing compound is chosen from aliphatic and aromatic hydrocarbon-based tackifying resins.

57. The composition according to claim 7, wherein the at least one medium is chosen from monolayers and multilayers of amphiphilic molecules that are solid at room temperature, and layers of polymers.

58. The composition according to claim 7, wherein the at least one medium is chosen from:
monolayers and multilayers of monomer and polymer surfactants, a portion of which is aliphatic and solid at room temperature,
a polymer shell,
a stack of at least two lipid bilayers, and
a liquid medium and two interfaces of monolayers of amphiphilic molecules.

59. The composition according to claim 7, wherein the at least one pro-adhesive material is in a dispersed form.

60. The composition according to claim 7, further comprising at least one cosmetically acceptable support.

61. A method for making a composition chosen from cosmetic and physiological compositions, comprising including in the composition
i) at least one pro-adhesive material that satisfies the following condition, for any frequency ranging from 2×10⁻² to 2 Hz 10⁸ Pa≧G′(35° C.)≧2×10⁴ Pa,
wherein:
G′(35° C.) is the elastic shear modulus of said at least one pro-adhesive material, measured at a temperature of 35° C.;
ii) at least one diffusing compound,
wherein a system created by the interaction of the at least one pro-adhesive material and the at least one diffusing compound is an adhesive system that satisfies the following conditions:
G′_{AS}(2 Hz, 35° C.)≧10³ Pa, and
G′_{AS}(2×10⁻² Hz, 35° C.)≦3×10⁵ Pa,
wherein:
G′_{AS}(2 Hz, 35° C.) is the elastic shear modulus of the adhesive system, measured at a frequency of 2 Hz and at a temperature of 35° C., and
G′_{AS}(2×10⁻² Hz, 35° C.) is the elastic shear modulus of the adhesive system, measured at a frequency of 2×10⁻² Hz and at a temperature of 35° C.; and
iii) at least one medium for separating the at least one pro-adhesive material and the at least one diffusing compound, wherein the at least one medium limits the molecular diffusion between the at least one pro-adhesive material and the at least one diffusing compound within the composition, and wherein the weight ratio between the at least one pro-adhesive material and the at least one diffusing compound ranges from 0.01:1 to 10:1.

62. A process for achieving at least one effect chosen from reduction and prevention of the transfer and the migration of a composition chosen from cosmetic and physiological compositions deposited on a keratin material towards a support placed in contact with the deposit, and enhancement of the staying power of the makeup and/or care result over time of the composition, comprising including in the composition
i) at least one pro-adhesive material that satisfies the following condition, for any frequency ranging from 2×10⁻² to 2 Hz 10⁸ Pa≧G′(35° C.)≧2×10⁴ Pa,
wherein:
G′(35° C.) is the elastic shear modulus of said at least one pro-adhesive material, measured at a temperature of 35° C.;
ii) at least one diffusing compound,
wherein a system created by the interaction of the at least one pro-adhesive material and the at least one diffusing compound is-an adhesive system that satisfies the following conditions:
G′_{AS}(2 Hz, 35° C.)≧10³ Pa, and
G′_{AS}(2×10⁻² Hz, 35° C.)≦3×10⁵ Pa,
wherein:
G′_{AS}(2 Hz, 35° C.) is the elastic shear modulus of the adhesive system, measured at a frequency of 2 Hz and at a temperature of 35° C., and
G′_{AS}(²×1 Hz, 35° C.) is the elastic shear modulus of the adhesive system, measured at a frequency of 2×10⁻² Hz and at a temperature of 35° C.; and
iii) at least one medium for separating the at least one pro-adhesive material and the at least one diffusing compound, wherein the at least one medium limits the molecular diffusion between the at least one pro-adhesive material and the at least one diffusing compound within the composition, and wherein the weight ratio between the at least one pro-adhesive material and the at least one diffusing compound ranges from 0.01:1 to 10:1.

63. A method for making a pulverulent composition chosen from pulverulent cosmetic and physiological compositions, comprising including in the composition
i) at least one pro-adhesive material that satisfies the following condition, for any frequency ranging from 2×10⁻² to 2 Hz 10⁸ Pa≧G′(35° C.)≧2×10⁴ Pa,
wherein:
G′(35° C.) is the elastic shear modulus of said at least one pro-adhesive material, measured at a temperature of 35° C.;
ii) at least one diffusing compound,
wherein a system created by the interaction of the at least one pro-adhesive material and the at least one diffusing compound is an adhesive system that satisfies the following conditions:
G′_{AS}(2 Hz, 35° C.)≧10³ Pa, and
G′_{AS}(2×10⁻² Hz, 35° C.)≦3×10⁵ Pa,
wherein:
G′_{AS}(2 Hz, 35° C.) is the elastic shear modulus of the adhesive system, measured at a frequency of 2 Hz and at a temperature of 35° C., and
G′_{AS}(2×10⁻² Hz, 35° C.) is the elastic shear modulus of the adhesive system, measured at a frequency of 2×10⁻² Hz and at a temperature of 35° C.; and
iii) at least one medium for separating the at least one pro-adhesive material and the at least one diffusing compound, wherein the at least one medium limits the molecular diffusion between the at least one pro-adhesive material and the at least one diffusing compound within the composition, and wherein the weight ratio between the at least one pro-adhesive material and the at least one diffusing compound ranges from 0.01:1 to 10:1, to increase the colour fastness of the composition over time.

64. A method of enhancing the colour fastness of a pulverulent composition chosen from pulverulent cosmetic and physiological compositions over time, comprising including in the composition
i) at least one pro-adhesive material that satisfies the following condition, for any frequency ranging from 2×10⁻² to 2 Hz 10⁸ Pa≧G′(35° C.)≧2×10⁴ Pa,
wherein:
G′(35° C.) is the elastic shear modulus of said at least one pro-adhesive material, measured at a temperature of 35° C.;
ii) at least one diffusing compound,
wherein a system created by the interaction of the at least one pro-adhesive material and the at least one diffusing compound is an adhesive system that satisfies the following conditions:
G′_{AS}(2 Hz, 35° C.)≧10³ Pa, and
G′_{AS}(2×10⁻² Hz, 35° C.)≦3×10⁵ Pa,
wherein:
G′_{AS}(2 Hz, 35° C.) is the elastic shear modulus of the adhesive system, measured at a frequency of 2 Hz and at a temperature of 35° C., and
G′_{AS}(2×10⁻² Hz, 35° C.) is the elastic shear modulus of the adhesive system, measured at a frequency of 2×10⁻² Hz and at a temperature of 35° C.; and
iii) at least one medium for separating the at least one pro-adhesive material and the at least one diffusing compound, wherein the at least one medium limits the molecular diffusion between the at least one pro-adhesive material and the at least one diffusing compound within the composition, and wherein the weight ratio between the at least one pro-adhesive material and the at least one diffusing compound ranges from 0.01:1 to 10:1.

65. The composition according to claim 1, wherein the composition is chosen from pulverulent cosmetic and physiological compositions.

66. The composition according to claim 7, wherein the composition is chosen from pulverulent cosmetic and physiological compositions.

67. A process chosen from non-therapeutic care and makeup processes for a keratin material, comprising applying to said keratin material a composition comprising
i) at least one pro-adhesive material that satisfies the following condition, for any frequency ranging from 2×10⁻² to 2 Hz 10⁸ Pa≧G′(35° C.)≧2×10⁴ Pa,
wherein:
G′(35° C.) is the elastic shear modulus of said at least one pro-adhesive material, measured at a temperature of 35° C.;
ii) at least one diffusing compound,
wherein a system created by the interaction of the at least one pro-adhesive material and the at least one diffusing compound is an adhesive system that satisfies the following conditions:
G′_{AS}(2 Hz, 35° C.)≧10³ Pa, and
G′_{AS}(2×10⁻² Hz, 35° C.)≦3×10⁵ Pa,
wherein:
G′_{AS}(2 Hz, 35° C.) is the elastic shear modulus of the adhesive system, measured at a frequency of 2 Hz and at a temperature of 35° C., and
G′_{AS}(2×10⁻² Hz, 35° C.) is the elastic shear modulus of the adhesive system, measured at a frequency of 2×10⁻² Hz and at a temperature of 35° C.; and
iii) at least one medium for separating the at least one pro-adhesive material and the at least one diffusing compound, wherein the at least one medium limits the molecular diffusion between the at least one pro-adhesive material and the at least one diffusing compound within the composition, and wherein the weight ratio between the at least one pro-adhesive material and the at least one diffusing compound ranges from 0.01:1 to 10:1.
